(19) United States
(12) Patent Application Publication
(10) Pub. No.: US 2023/0190376 A1
(43) Pub. Date: Jun. 22, 2023

(54) Surgical System

(71) Applicant: Formus Labs Limited, Auckland (AU)

(72) Inventors: Andrew Paul Monk, Auckland (NZ); Thor Franciscus Besier, Auckland (NZ); Mousa Kazemi, Auckland (NZ); Ju Zhang, Auckland (NZ)

(21) Appl. No.: 17/967,701

(22) Filed: Oct. 17, 2022

Related U.S. Application Data
(63) Continuation-in-part of application No. PCT/NZ2021/050062, filed on Apr. 12, 2021.

(30) Foreign Application Priority Data
Apr. 20, 2020 (NZ) 763680
Apr. 21, 2020 (NZ) 763731

(51) Int. Cl. A61B 34/10; A61B 34/00; G06T 19/20

(52) U.S. Cl. CPC A61B 34/10; A61B 34/25; G06T 19/20; A61B 2017/568; A61B 2034/105; A61B 2034/108

(57) ABSTRACT

A removable wedge for high tibial osteotomy surgery, comprising: a truncated wedge configured to provide a patient specific correction to the weight bearing axis based on 3D data for that patient’s tibia, femur and/or fibula; and an anterior flange configured to locate the partial wedge in a predetermined location on the tibia of that patient. Also a method for pre-operative planning, a method of designing a wedge, a method of printing a wedge a reusable wedge and a prosthetic implant.

### CROSS-REFERENCE TO RELATED APPLICATIONS

This application is a bypass continuation of International PCT Patent Application No. PCT/NZ2021/050062 filed on Apr. 12, 2021, which claims priority New Zealand Patent Application No. 763680 filed on Apr. 20, 2020, and New Zealand Patent Application No. 763731 filed on Apr. 21, 2020, which are incorporated by reference herein in their entirety.

### FIELD

This invention relates to a surgical system.

### BACKGROUND

FIG. 1 illustrates a patient with bowlegged (Varus deformity) 102 or knock kneed (Valgus deformity) 104. It is known to provide a tibial correction by way of a high tibial osteotomy (HTO) to treat either of these conditions.

For example, in U.S. Pat. Publication 2018344371 a system for HTO was disclosed including a removable shim, a wedge prosthesis and a support plate.

### SUMMARY

According to one example embodiment there is provided a removable wedge for high tibial osteotomy surgery according to independent claim 1, a method of designing a wedge for high tibial osteotomy surgery according to independent claim 9, a process for manufacturing a wedge for high tibial osteotomy surgery according to independent claim 10, a method for pre-operative planning of a high tibial osteotomy surgery according to independent claim 11 or 28, a reusable wedge according to independent claim 23 or a prosthetic implant according to independent claim 26.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning – i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings which are incorporated in and constitute part of the specification, illustrate embodiments of the invention and, together with the general description of the invention given above, and the detailed description of embodiments given below, serve to explain the principles of the invention, in which:

FIG. 1 is a front view of patient deformities;

FIG. 2 is a schematic diagram of an example knee joint;

FIG. 3 is a 3D model of a tibial plateau;

FIGS. 4 and 5 are 3D models of two cuts required for a HTO treatment;

FIG. 6 is a flow diagram of a method of HTO treatment according to an example embodiment;

FIG. 7 is an example user interface for pre-operative planning;

FIG. 8 is a 3D model of a patient specific wedge for the treatment in FIG. 6;

FIG. 9 is a block diagram of a system for pre-operative planning according to an example embodiment;

FIG. 10 is a schematic diagram of a reusable wedge according to an alternative embodiment; and

FIGS. 11A and 11B are schematic diagrams of an integrated wedge and support plate according to a further alternative embodiment.

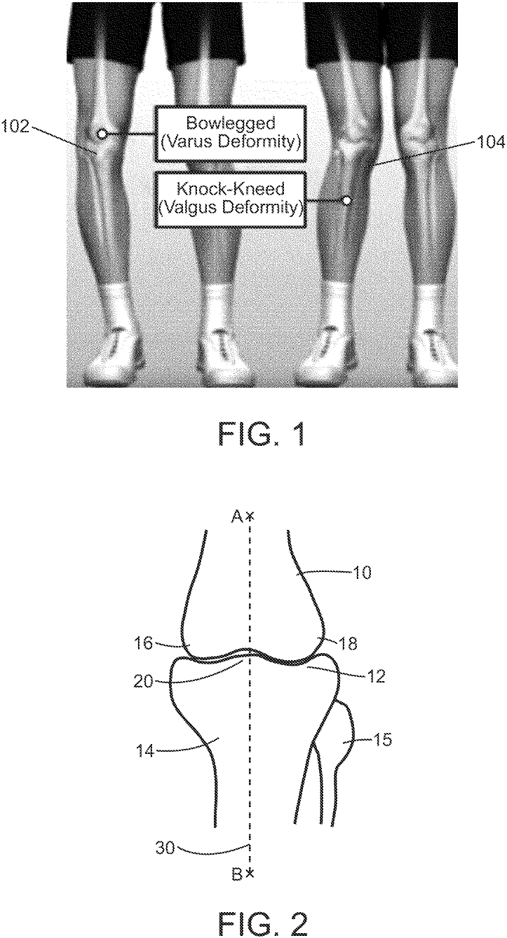

### DETAILED DESCRIPTION

In general terms, one or more embodiments may relate to a custom wedge for HTO, and/or an implementation of pre-operative planning software for HTO surgery, where the wedge parameters are optimised for each patient from functional / task simulations and/or using a deformable model for soft tissues.

The wedge may have the advantage that it can be inserted to precisely define the corrected position while a support plate is attached (no need for pinning but could be), then removed, which may allow fixing of the support plate to be more accurate than with a removable shim or wedge prosthesis that is not locked in place in a patient specific location. The wedge may also form part of the support plate itself.

The pre-operative planning software may have the advantage that it is numerically stable and/or able to provide an effective and efficient decision-making tool for deciding the sagittal wedge angle and the coronal wedge angle based on loading data, heatmaps, pressures graphs, and/or visual constructs of corrections with weightbearing line, mechanical axis. The software may display pressure maps along-side 3D models of the leg virtually corrected by a wedge of a user selected angle. This may allow the user to visualize the correction to be done. The software may also automate parts of or the whole process from image processing to simulations.

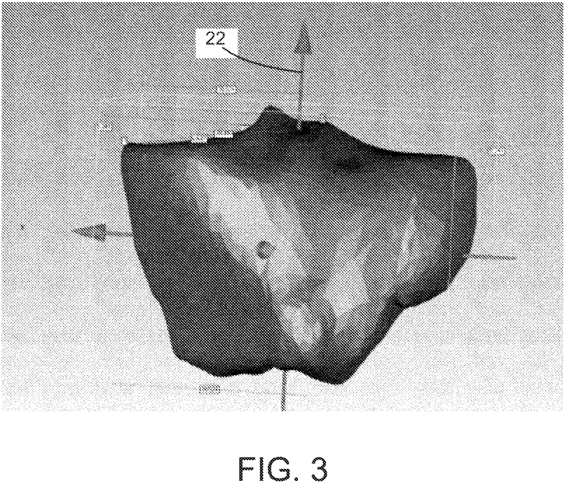

Referring to FIG. 2, the knee joint is where the distal end of the femur 10 rests on the tibial plateau 12 at the proximal end of the tibia 14. The upper end of the fibula 15 is below the tibial plateau 12. The medial and lateral condyles 16, 18 of the femur each bear on the superior surface of the tibial plateau. If the alignment between the femur 10 and tibia 14 is correct, the loading on the two condyles 16, 18 is approximately equal, and the direction of loading is vertically through the intercondylar eminence 20 in the centre of the tibial plateau 12, i.e. in the direction of the arrow 22 shown in FIG. 3. If the alignment of the knee moves away from the ideal, for example in a varus knee, the loading in the knee becomes uneven and various complications arise.

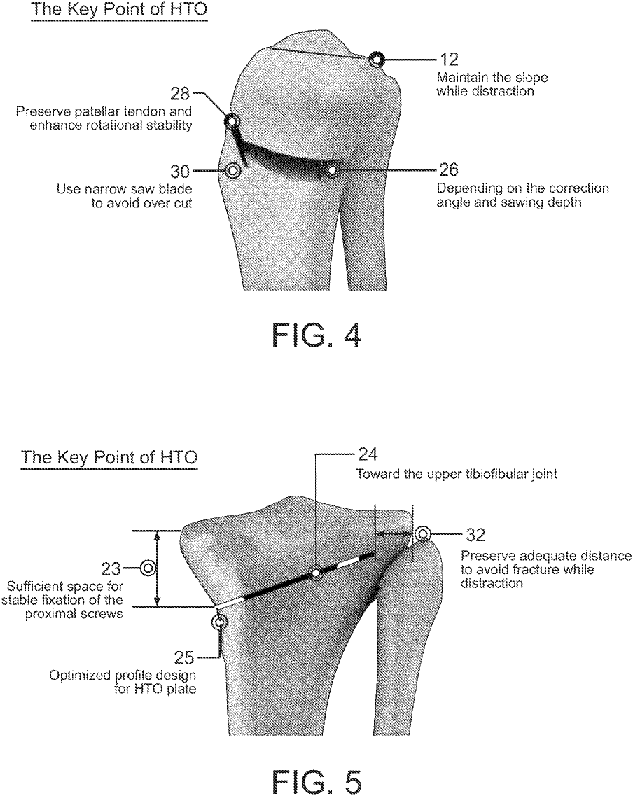

Referring to FIGS. 4 and 5, one method of treatment of a varus knee is a high tibial osteotomy, in which the orientation of the top of the tibial plateau 12 is adjusted by making a first cut 24 through the tibia 14 at a depth 23 below the plateau 12, in this case from the medial side in the lateral direction and slightly upwards, and then opening the cut 24 to form a wedge shaped gap 26. The two parts of the tibia on either side of the cut can then be secured in place relative to each other, for example using a securing member such as a plate extending across the open end of the gap 26 and secured to the bone on either side. The depth 23 may be selected to give sufficient space for suitable fixation of proximal screws as well as the optimised location for the support plate on the medial part of the proximal tibia 25. Osteotomy can be performed on other bones, such as in the hip, to address other problems, and that a tibial osteotomy is only one example. While for a varus deformity, the correction may be an opening medial wedge or closing lateral wedge, conversely for a valgus deformity, the correction may be an opening lateral or closing medial.

A second cut 28 (bi-planar osteotomy) can also be included, of width 30, to preserve the attachment of the patella tendon, using a narrow saw blade to avoid overcutting. Alternatively, the osteotomy may be performed below the level of the tibial tubercle.

The exact position, size and orientation of the gap 26 will determine the final orientation of the top of the tibial plateau 12, and hence the load distribution in the tibia and femur and the final orientation of the tibia relative to the femur.

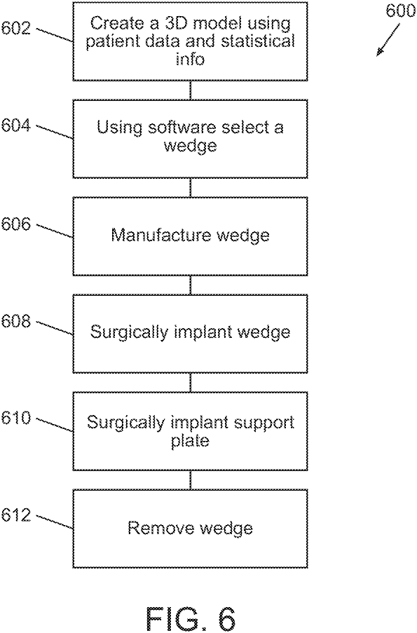

Referring to FIG. 6, a method 600 of high tibial osteotomy is shown according to an example embodiment. A 3D model is created 602 using patient specific anthropometric data and/or statistical information. The 3D model is then used by medical specialists to select 604 a desired HTO wedge angle. A patient specific wedge is manufactured 606 according to the directed angle. The manufactured wedge is inserted 608 during surgery, a support plate is inserted 610, and the wedge is removed 612.

### Pre-Operative Planning

### 3D Model

In order to model the contact pressures by various offloading angles, a 3D model of the knee is required, plus:
a 3D model of the proximal femur and distal tibia,
Length of the femur and length of the tibia, or
Location of the hip joint centre and the ankle joint centre

The model may also include the Femur and distal femur cartilage and the tibia and proximal tibia cartilage. Depending on the requirements of the application the model may also include the meniscus, the anterior cruciate ligament, the posterior cruciate ligament, the medial collateral ligament, the lateral collateral ligament, the patella and patella cartilage, and/or one or more muscles or other soft tissues.

The 3D Model may include the surface geometry of the entire tibia and fibula, which may be represented by a triangulated mesh (but other representations may be used according to the application requirements, e.g. b-splines, non-uniform rational basis spline (NURBS)). The 3D model of the bones and cartilage are created from the MRI. This can be through manual segmentation or automatic segmentation. Automatic segmentation can be via a series of image filters like thresholding, region-growing, and edge detection. It can also be through a model-based method such as an active shape model, active appearance model, or a convolutional neural network.

The models produced above are typically of portions of bones since the field-of-view of each scan only covers a joint. For example, a knee MRI scan may produce 3D models of the distal femur and the proximal tibia, but not of the whole femur or whole tibia. The whole-bone geometries are required to simulation the kinematics of the whole limb.

To obtain models of the whole bones, statistical shape models (SSM) are used to reconstruct whole-bone models from the partial bone models, magnetic resonance imaging (MRI) scans of the hip, knee, and ankle are obtained a low, high, and low resolutions, respectively. Partial 3D models of the femur, tibia, and fibula are segmented from the scans. For the femur, a mean femur model is morphed to fit to the partial femur 3D models through optimisation of the model’s position, orientation, and shape as parameterised by the SSM. After this morph, a finer-scale morph is performed at the proximal and distal femur regions using a local morphing method. A similar process is performed for the tibia and fibula. FE model generation using morphing and region mapping methods may allow the process to be unsupervised and / or automated. If medium or higher resolution scans are obtained for the hip and ankle, femur and tibia models can be constructed from the segmentations directly without using a shape model. The models would consist of segmented proximal and distal ends, with interpolated triangles spanning the space in between (the diaphysis of the bones). We do not need highly accurate diaphysis geometry because the subsequent finite element modelling is not concerned with the diaphysial region. Low res scans: >=10 mm slice spacing, medium res scans: ~3 mm slice spacing, and high res scans : ~1 mm slice spacing.

The whole-bone 3D models are then aligned to the patient’s weight bearing (WB) X-ray to represent their neutral (standing) pose and reconstruct their knee mechanical and anatomical axes. One way of performing the alignment is to
1) manually or automatically detected the bone outline from the X-rays and magnify by an amount indicated by calibration markers in the X-ray image.
2) If there is more than 1 X-ray, they should be at right angles to each other and their outlines should also be aligned to be at right angles to each other
3) optimise the position and orientation of the 3D models to fit to the outline(s).

The registered 3D models are then articulated according to knee joint angles calculated from motion capture. The joint angles may simulate walking, Sit to stand, Squat to stand, stair climb and descending, Jogging/running, side-step and/or other sport-specific motions or tasks. Motion capture (such as optical mo-cap) may identify the knee joint angles, or they can be simulated by performing these activities using a database or statistical model of body motion. Simulation

After alignment, the 3D models are used to generate a finite element (FE) model of the knee. In a rigid-body model of the knee, the 3D models are used as is (surface models). In a deformable model of the knee, the 3D models are converted into volumetric meshes with either tetrahedral or hexahedral elements. Boundary conditions and constraints are then mapped onto points or regions of the meshes to simulate mechanical loads (e.g. body weight, muscle forces, and ground reaction force), contact (between bones, cartilage layers, the meniscus), and mechanical constraints (e.g. ligaments, meniscus). In general, the tibia and fibular are fixed in position and orientation while the femur is free to move while a force (e.g. half body-weight while standing) is applied at the femoral head. The geometric configuration of the FE model is modified for each wedge angle by altering the direction of load at the femoral head to efficiently simulate the change in mechanical axis resulting from the insertion of a wedge. Alternatively, we can fix the femur and leave the tibia and fibula free to move, depending on the surgeon’s preference. Also, the forces can be applied at the bone centre of mass as a rigid-body force to further simplify the simulation.

The morphed mesh has the same mesh topology for every patient. Therefore, the anatomical points and regions can be defined once on the mean mesh in terms of their vertex and face indices and know where they are on any morphed patient mesh. This allows boundary conditions to be automatically assigned, loads to be automatically assigned, and other constraints on the relevant points and regions of the mesh to be automatically assigned. If a shape model was not used in the 3D Modelling step, the points and regions can still be defined manually.

Further details of the process of morphing and region mapping are provided in copending New Zealand patent application number 763679, entitled “Orthopaedic Pre-Operative Planning Software”, filed by the same Applicant as the present application on 20 Apr. 2020, the contents of which are incorporated herein by reference.

The locations of the osteotomy entry and hinge points are defined on the FE model with input from the surgeon. In the planning software, the user can click these points through the user interface, or the software can define them automatically based on heuristics about their standard positions.

The FE simulation is run for a range of wedge size and angles to generate pressure maps from which an optimal set of wedge properties can be determined automatically or by a surgeon.

A maj or challenge of FE modelling of musculoskeletal system is the numerical stability of the model, and its computational performance. Both tend to decrease as the fidelity of the model increases, especially in a deformable FE model. Significant improvements in stability and performance can be made by using a rigid-body model that allows the simulation to be run automatically in minutes rather than with manual adjustments over hours or days.

An implementation of an appropriate rigid-body model uses tension-compression contact modelling to estimate relative pressure between the medial and lateral compartments of the knee. The rigid-body model may have far fewer degrees of freedom than a fully deformable model and so may solves faster or be better conditioned numerically. It may require no manual tuning for the simulation to solve, whereas a deformable model may require days of tuning. Note that the goal of the simulation is to determine how the wedge angle changes the relative loading of the compartments. Therefore, the absolute pressure is not important.

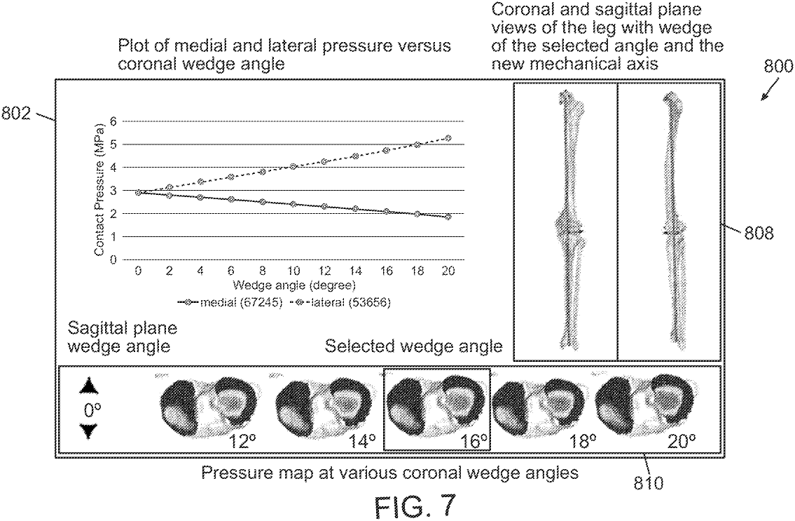

FIG. 7 shows an example user interface 800 to select the optimum (HTO) correction plan. The software will make a recommended correction, but the user (surgeon) may change that selection based on reviewing the pressure graphs shown.

The criteria for the suggested correction could be:
The minimum angles at which the medial compartment is completely offloaded at standing
The minimum angle at which the medical compartment is completely offloaded through the gait cycle (or some other functional task)

On the left hand top area of the screen 800 is a chart 802 of the peak, mean, or total pressure (force) in the medial 804 and lateral 806 tibial compartment versus coronal wedge angle. The user can also alternatively select the pressure chart for sagittal wedge angles at a given coronal angle

On the right hand side, a 3D model 808 is shown of a fixed front-on view of the leg showing the native and post-op mechanical axis. This also shows the planned wedge, femur, tibial, and cartilage on each bone, plus the other soft tissue structures if available, focused on the knee. As the user selects different wedge angles, the wedge model changes along with the knee geometry. The tibia below the wedge is fixed while the tibia above the wedge and the femur (plus soft tissue) pivots according to the wedge.

Below the chart 802 is a series of 3D pressure maps 810 for a range of different coronal angles for the selected sagittal angle. This panel 810 can be expanded upwards to show a grid of all pressure maps for all coronal and sagittal angles. In the expanded view, the user can zoom in and out from the full grid to a particular pressure map. Selecting a pressure map will update the selected angles and the models in the 3D scene.

### Manufacturing the Wedge

Sending the final model for designing wedge to a 3D printer may be done as described below.

The 3D HTO wedge angle is designed as above, then FE model wedge shape and the parts (wedge, plate, screws, ...) are determined in order to achieve the desired wedge angle in terms of a practical surgical plan. Solidworks may be used to design the wedge, based on the FE model results. Lastly the wedge and support plate may be 3D printed using Dental SG resin. The 3D printer may be provided offsite or at the surgery.

### The Wedge

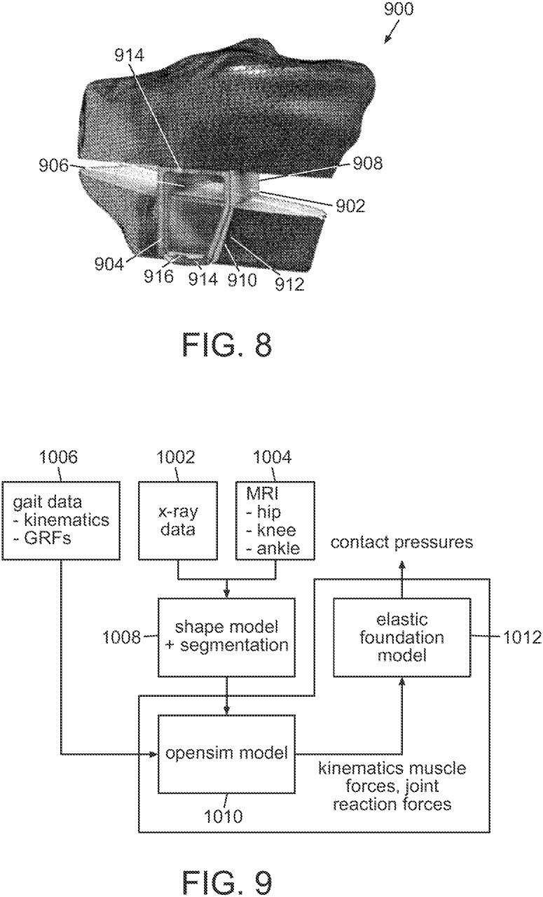

An example wedge 900 is shown in FIG. 8. The wedge 900 includes a truncated wedge 902 and an anterior flange 904. The partial wedge 902 is hollow, having an open anterior end 906 and a closed posterior end 908. The partial wedge 902 is truncated because it is inserted from the anterior side, parallel to the hinge axis, and has a relatively narrow width. The shape if the wedge is designed so that it can fit between the support plate and the patella tendon. The full wedge is inserted in silico on the planning model and virtual surgery performed. The location of the tendon and position of the plate are superimposed and the space available for the wedge identified. The wedge is trimmed to the appropriate shape.

The anterior flange 904 is included to determine the wedge position within the first cut, and to allow effective insertion and withdrawal. A posterior face 910 of the flange 904 is designed to conform to the geometry of the anterior tibia 912. In particular, the posterior face 910 should mould over the tibial tuberosity. The flange 904 includes 2 tabs 914, and each tab includes a hole 916. As described later, the holes 916 may be used for insertion and/or removal during surgery.

The wedge may be 3D printed on a Formlabs 3D printer using Dental SG resin. This allows it to be sterilised in an autoclave. Alternatively, it may be printed or milled from plastic, nylon, metal, bone, or any combination thereof.

The anterior flange shape is generated from the Boolean subtraction of the tibia geometry from a solid extrusion of the wedge 20-30 mm inferiorly into the tibia.

### Reuseable Wedge

A reusable wedge may also be employed that is adjustable to desired angles in the coronal and sagittal planes.

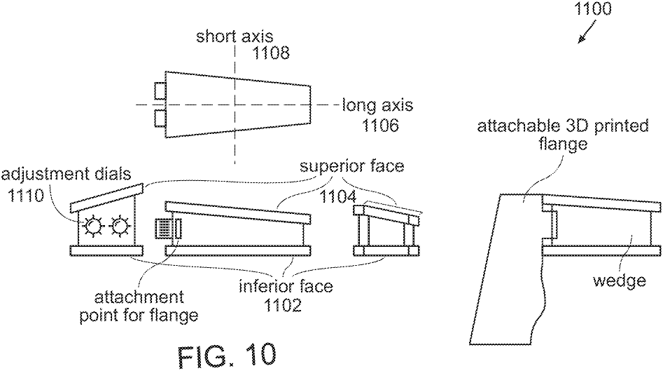

As shown in FIG. 10, the wedge 1100 can be manufactured from stainless-steel parts including a flat inferior face or plate 1102, a flat superior face or plate 1104, an internal ratchet system that changes the superior face angle relative to the inferior face about the wedge’s long 1106 and short axes 1108, external dials 1110 that adjust the ratchet system and therefore the wedge angle. The ratchet prevents the wedge angle changing once angles have been configured. The wedge 1100 could be configured by turning dials to marked angle positions on the exterior of the wedge. The wedge could be configured prior to insertion into the bone cut or after insertion into the bone cut (dial the angle up to the desired value).

The wedge could also be adjusted automatically and wirelessly. In this case, the wedge could contain an internal wireless communication module (e.g. Bluetooth), power supply (e.g. wireless rechargeable battery), actuators that adjust the wedge angles, sensors to measure the wedge’s current angles and a controller to drive the actuators to a predetermined coronal and/or sagittal correction. In this case, the wedge can be configured directly from the planning software running on a computer with a compatible wireless communication module (e.g. Bluetooth). The wedge would communication its current angles back to the planning software to confirm that it has been correctly configured.

The wedge could also contain load-cells to measure the force being exerted on its superior and inferior faces. This is useful to prevent breaking the bone by using imposing wedge angles that are too large. A possible use case is when the wedge is inserted into the bone cut in its lowest angles configuration then adjusted up to the desired angles. As the angle is incrementally increased, the wedge can transmit the force it is experiencing to the software which displays the value to the user. If the force exceeds a threshold, a graphical and/or audio warning is emitted by the software and/or the wedge.

### Integrated Wedge and Support Plate

The wedge could also be made of a bio-absorbable or integrable material, e.g. bone allograft. In this case, the wedge would be a permanent implant left in the patient’s body. Such a wedge would incorporate with the bone.

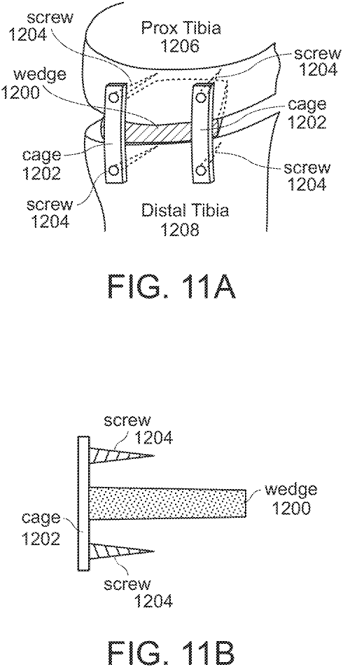

Using such a wedge would avoid having to use a plate to fix the bone and act as mechanical support. As shown in FIGS. 11A and 11B the wedge 1200 itself would be the load-bearing structure that may be supported by a cage 1202 attached to the wedge and fixed by screws 1204 to the proximal 1206 and distal 1208 portions of the tibia. The wedge 1200 and cage / support plate 1202 may be integral.

### HTO Surgery

As mentioned above Virtual reality (VR) allows the HTO operation to be practiced using the previous models. Similarly, during the operation, using the patient’s real-time image processing (registration of the 3D models on lower limb of the patient during the operation) the location of the implants can be matched against the surgical plan.

Once the cuts are made, several holes are drilled in the cortex/lateral hinge, to reduce the likelihood of a fracture. Additionally, the depth 32 of the first cut 24 may be adjusted, to reduce the likelihood of a fracture in the cortex/lateral hinge. The depth 32 of the first cut 24 may finish 1 cm from the cortex. It may be controlled by the slow introduction of stacked osteotomes.

The wedge is inserted anteromedially, reflecting the medial collateral ligament posteriorly with a retractor. The support plate is inserted attached using sequential screws (locking and non-locking). The support plate may be a Tomofix® support plate marketed by DePuySythes. The Tomofix® may be surgically inserted according to the technique annexed hereto.

The 2 holes are used as a point of attachment for a tool to hold and pull or push wedge during insertion and retraction.

### Software System

Referring to FIG. 9, a system 1000 for preoperative planning is shown according to an example embodiment. This system 1000 may be executed on a cloud based or local server or workstation. The user may access the system 1000, by authenticating on a user interface (UI) such as a https web browser connection.

The system 1000 includes a data store for the X-Ray data 1002, the MRI data 1004, and the gait data 1006. The X-Ray data 1002 and the MRI data 1004 is used to construct the shape model and segmentation data 1008. The shape model and segmentation data 1008 and gait data 1006 is used to construct the opensim model 1010, which calculates the kinematics, muscle forces and joint reaction force to generate an elastic foundation model 1012. The elastic foundation model 1012 may then be used to simulate the 3D contact pressure graphs.

Using a UI, the user may initially create a case, then upload the MRI and X-Ray data together with patient details such as patient height. The MRI data may have a minimum of 5-mm spacing and 5-mm thickness in the hip and ankle, 0.5 mm spacing and thickness in the knee and with a 150-mm range centre on the knee joint.

Image segmentation may occur automatically or may involve user intervention.

Templating occurs through the generation and running of FE models of the knee at a range of wedge angles to generate pressure maps of the knee at each wedge angle. This may occur automatically or may involve user intervention.

Once the 3D model is complete the system is then free to generate reports. For example the UI described earlier in relation to FIG. 7 may be used by the user to review a series of pressure maps corresponding to various wedge angles. The use may select a specific wedge angle by clicking on a pressure map or by directly entering a wedge angle. Also displayed is one or more 3D presentation of the leg (e.g. from different view points) after the selected wedge is applied.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant’s general inventive concept.

1-28. (canceled)

29. A method for pre-operative planning of a high tibial osteotomy surgery, comprising:
storing patient data including a 3D model representing at least a substantial portion of the tibia, femur and/or fibula of a first patient, and motion analysis data for at least the knee joint for the first patient;
simulating 3D contact pressure graphs for the patient for a range of coronal and sagittal corrections and/or in a range of motions or tasks based on the 3D model and the motion analysis data; and
selecting an optimised high tibial osteotomy correction for the first patient;
wherein the 3D model includes a whole-bone model of each of the tibia, femur and/or fibula.

30. The method of claim 29, further comprising reconstructing the whole-bone model from a partial-bone model.

31. The method of claim 30, wherein the whole-bone model is reconstructed by morphing a mean bone model to fit the partial bone model.

32. The method of claim 29, wherein the 3D model includes elements selected from the group consisting of bones, cartilage, ligaments, meniscus, muscles, and any combination thereof.

33. The method of claim 32, wherein the 3D model includes a rigid body model of one or more of the cartilage, ligaments, meniscus, and/or muscles.

34. The method of claim 29, wherein the 3D model includes an elastic deformable model of one or more soft tissues.

35. The method of claim 29, wherein the 3D model includes a rigid body model of the tibia, femur and/or fibula.

36. The method of claim 29, further comprising segmenting MRI data for the first patient to construct the 3D model.

37. The method of claim 36, further comprising finer-scale morphing at the proximal and distal femur regions using a local morphing method.

38. The method of claim 37, further comprising region mapping including automatically assigning boundary conditions and/or load to each region.

39. The method of claim 36, further comprising templating the segmented data to construct the 3D model.

40. The method of claim 39, further comprising aligning the 3D model with a standing X-Ray of the first patient.

41. The method of claim 29, further comprising articulating the 3D model according to knee joint angles calculated from the motion analysis data.

42. The method of claim 29, wherein the 3D model includes the knee joint and a selection from the group consisting of a 3D model of the proximal femur and distal tibia, the length of the femur and length of the tibia, and a location of the hip joint centre and the ankle joint centre.

43. The method of claim 29, further comprising selecting a patient specific wedge based on the optimised high tibial osteotomy correction for the first patient.

44. The method of claim 29, further comprising using tension-compression contact modelling to estimate relative pressure between compartments of the knee.

45. A method for pre-operative planning of a high tibial osteotomy surgery, comprising:
storing patient data including a 3D model representing at least a rigid body model of a substantial portion of the tibia, femur and/or fibula of a first patient;
simulating 3D contact pressure graphs for the patient for a range of coronal and sagittal corrections and/or in a range of tasks; and
selecting an optimised high tibial osteotomy correction for the first patient.

46. The method of claim 45, wherein the 3D model includes a whole-bone model of each of the tibia, femur and/or fibula.

47. The method of claim 46, wherein the whole-bone model is reconstructed by morphing a mean bone model to fit a partial bone model.

48. The method of claim 45, wherein the 3D model includes a rigid body model of one or more of the cartilage, ligaments, meniscus, and/or muscles.

49. The method of claim 45, further comprising:
displaying the 3D model on a user interface;
receiving input from a user via the user interface; and
defining osteotomy entry and hinge points based on the received input.

50. The method of claim 45, further comprising:
displaying, on a user interface, 3D pressure maps for different coronal and sagittal angles;
receiving a selection of one of the 3D pressure maps via the user interface; and
generating an optimised high tibial osteotomy correction for the first patient using the selected 3D pressure map, the optimised high tibial osteotomy correction including updated coronal and sagittal angles.

51. The method of claim 45, further comprising:
displaying, on a user interface, a chart of pressures in medial and lateral tibial compartments at different coronal and sagittal wedge angles;
receiving a selection of at least one of the coronal or sagittal wedge angle via the user interface; and
generating an optimised high tibial osteotomy correction for the first patient using the selected wedge angle, the optimised high tibial osteotomy correction including updated coronal and sagittal angles.

52. The method of claim 45, further comprising:
generating a wedge design for a wedge using the optimised high tibial osteotomy correction for the first patient; and
sending the wedge design to a 3D printer.
